(19) United States
(12) Patent Application Publication
(10) Pub. No.: US 2024/0387012 A1
(43) Pub. Date: Nov. 21, 2024

(54) SYSTEM AND METHOD FOR USING EMR DATA FOR CLINICAL DECISION SUPPORT

(71) Applicant: KONINKLIJKE PHILIPS N.V., EINDHOVEN (NL)

(72) Inventors: MAHMOUDREZA SHARIFI, NEW ROCHELLE, NY (US); CORNELIS VAN ZON, EVERETT, MA (US)

(21) Appl. No.: 18/690,123

(22) PCT Filed: Sep. 2, 2022
(86) PCT No.: PCT/EP2022/074501

§ 371 Date: Mar. 7, 2024

Related U.S. Application Data
(60) Provisional application No. 63/241,153, filed on Sep. 7, 2021.

(51) Int. Cl. G16H 10/60

(52) U.S. Cl. CPC G16H 10/60

(57) ABSTRACT

Systems, apparatuses and methods provide for the management of electronic medical records (EMR) for clinical decision support (CDS) applications. For example, an apparatus (100) is configured to parse an EMR database (102), determine whether supplemental entry of current patient data is needed based on existing patient data in the EMR database, and transfer a notification that the supplemental entry of the current patient data is needed to a user interface (126) associated with a care provider.

### FIELD

The following relates generally to medical records. More particularly, embodiments herein relate to the use of electronic medical records (EMR) for clinical decision support (CDS).

### BACKGROUND

The use of EMR data for real-time decision making is still a challenge in clinical settings. The EMR system was originally designed for archiving the clinical data for insurance and liability purposes. Additionally, many entries of EMR data are done manually by clinicians.

There have been several government initiatives (e.g., Health Information Technology for Economic and Clinical Health Act (HITECH)) that have created incentives related to health care information technology. As a result, there have been efforts to develop solutions to make better use of the valuable resource of EMR data, e.g., for improvement of the patient outcome and reduction of healthcare cost.

Clinical decision support (CDS) refers to computer-based support of clinical staff responsible for making decisions for the care of patients. Computer-based support for clinical decision-making staff is widespread and can take many forms, from patient-specific visual/numeric health status indicators to patient-specific health status predictions and patient-specific health care recommendations. CDS has steadily been accepted into mainstream healthcare, and this may be due in part to CDS only providing decision-making support and not being used as a substitute for clinical staff decision-making.

There are several inherent problems with EMR systems. Accordingly, it is currently challenging to use EMR systems as a reliable source for real-time CDS algorithms.

The following discloses certain improvements to overcome these problems and others.

### SUMMARY

As discussed above, there are several inherent problems with EMR systems. Some of these are due to the fact that majority of data in EMR data is entered manually by operator or healthcare personnel. Accordingly, it is currently challenging to use EMR systems as a reliable source for real-time CDS algorithms.

When the EMR data entry process is not automatic and requires human interaction the first obvious problem is human error. These human errors are sometimes due to typos in medial notes and sometimes due to lack of standard terminology among healthcare facilities and/or even among clinicians in one hospital. For example, for ventilated patients, a ventilation mode may have been given a specific commercial name by medical device vendor vs. a more generic name used in respiratory terminology. In such a situation, human error may be due to nonstandard language.

In addition to these problems, when the EMR data entry process is not automated there may be delays between the time, e.g., a medical test is done to the time that the test result is available in the EMR system. One reason for such a delay is that manual data entry may not have high priority in the clinical workflow. In some cases, as long as the EMR data is entered in the EMR database appropriately, the clinician is often considered to have done their job.

Accordingly, a CDS application may be waiting for a piece of information from collected but unentered EMR data. However, there is typically no systematic way to inform the responsible clinician of this situation. As a result, the CDS application may be delayed in delivering its output or produce output that is not fully informed; this limits the usability of the CDS application in practice. Similarly, there is typically no systematic way to inform the responsible clinician that human EMR data entry error has adversely impacted clinical decision support.

As will be discussed in greater detail below, some embodiments described herein will minimize a delay and/or error in EMR data entry by generating an interaction between a CDS application and an associated care provider in charge of data entry. For example, a notification will be sent to the associated care provider in charge of data entry when a necessary piece of information is needed.

In some implementations herein, a method and system are presented to parse the EMR database, determine whether supplemental entry of current patient data is needed based on existing patient data in the EMR database, and transfer a notification (e.g., an alert, a reminder, and/or a specific time-line) to a user interface associated with a care provider (e.g., a nurse station, a device associated with a specific care provider, a patient monitor associated with a specific patient in question, a therapeutic device associated with the specific patient in question, the like, or combinations thereof).

In one aspect, an apparatus includes an electronic medical record parser and an electronic medical record dispatch engine communicatively coupled to the electronic medical record parser. The electronic medical record parser may be communicatively coupled to an electronic medical record database. The electronic medical record parser may determine whether supplemental entry of current patient data is needed based on existing patient data in the electronic medical record database. The electronic medical record dispatch engine may transfer a notification that the supplemental entry of the current patient data is needed to a user interface associated with a care provider.

In another aspect, a method includes determining, via an electronic medical record parser, whether supplemental entry of current patient data is needed based on existing patient data in an electronic medical record database. The method also includes transferring, via an electronic medical record dispatch engine, a notification that the supplemental entry of the current patient data is needed to a user interface associated with a care provider.

In yet another aspect, a machine-readable storage includes machine-readable instructions, which when executed, include operations to determine whether supplemental entry of current patient data is needed based on existing patient data in an electronic medical record database. The machine-readable instructions, which when executed, also include operations to transfer a notification that the supplemental entry of the current patient data is needed to a user interface associated with a care provider.

In still another aspect, an apparatus includes means for determining whether supplemental entry of current patient data is needed based on existing patient data in an electronic medical record database. The apparatus also includes means for transferring a notification that the supplemental entry of the current patient data is needed to a user interface associated with a care provider.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

The various advantages of the embodiments will become apparent to one skilled in the art by reading the following specification and appended claims, and by referencing the following drawings, in which:

FIG. 1 is an illustration of a block diagram of an example EMR management system according to an embodiment;

FIG. 2 is an illustration of a block diagram of a data entry system according to an embodiment;

FIG. 3 is an illustration of a flowchart of a method of operating an EMR management system according to an embodiment;

FIG. 4 is a further illustration of a flowchart of a method of operating an EMR management system according to an embodiment;

FIG. 5 is an illustration of a screen shot of a user interface according to an embodiment;

FIG. 6 is an illustration of a block diagram of a computer program product according to an embodiment;

FIG. 7 is a further illustration of a EMR management system according to an embodiment; and

FIG. 8 is an illustration of a hardware apparatus including a semiconductor package according to an embodiment.

### DETAILED DESCRIPTION

As will be described in greater detail below, in some implementations discussed herein, an interactive tool may be used to address parts of the challenges in using EMR system as a reliable source for real-time CDS. For example, such an interactive tool may be used to notify responsible operators and/or clinicians about patient information that is missing and/or overdue in the EMR system.

Such a notification may be generated based on one or more of several different criteria. For example, such a notification may be frequency based. In such an example a notification (e.g., an alert and/or a specific timeline) may be sent when certain data is missing after certain period of time. In another example, the notification may be sent when certain data is needed by at least one active application. In such an example, a frequency of supplying refreshing the data may also be set by the individual active applications. Additionally, or alternatively, the notification may be sent based on a forecasted need for new patient data and/or refreshed patient data. In such an example, the forecasted need for new patient data and/or refreshed patient may be based on one or more algorithms. These algorithms may determine that a particular CDS algorithm currently needs such patient data and/or forecast that the particular CDS algorithm will need such patient data in the future. For example, a blood gas estimation-type algorithm may determine an estimation error progress overtime. When such an estimation error is beyond some value (e.g., a threshold value), a request for new patient data and/or refreshed patient data is generated.

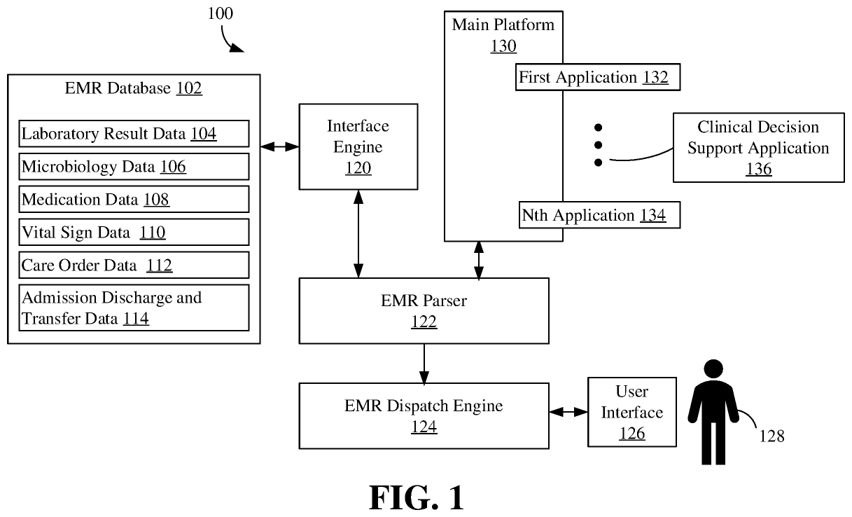

FIG. 1 is an illustration of a block diagram of an example EMR management system 100 according to an embodiment. For example, the EMR management system 100 may be centralized or may be distributed and may include some or all elements and components of one or more computers or computer systems. In the illustrated implementation, the EMR management system 100 may include an electronic medical record parser 122 coupled to an electronic medical record database 102, and an electronic medical record dispatch engine 124 communicatively coupled to the electronic medical record parser 122.

In the illustrated implementation, the electronic medical record database 102 may include one or more types of patient data. For example, the electronic medical record database 102 may include laboratory result data 104, microbiology data 106, medication data 108, vital sign data 110, care order data 112, admission discharge and transfer data 114, the like, and/or combinations thereof.

As used herein, the term “database” refers to a collection of data and information organized in such a way as to allow the data and information to be stored, retrieved, updated, and/or manipulated. The term “database” as used herein may also refer to databases that may reside locally or that may be accessed from a remote location (e.g., via remote network servers).

As used herein, the term “patient data” refers to data or information for identifying an individual. Patient data may include measured patient data from an analog medical device, a patient monitor, a therapeutic device, a medical management device, a medical imaging device, the like, and/or combinations thereof. Additionally, patient data may include a patient's name, age, weight, previous medical history, admission number, medical personnel in charge, date of admission, medical condition, a medical status, like, and/or combinations thereof.

In the illustrated implementation, the EMR management system 100 may also include an interface engine 120. The electronic medical record parser 122 may be coupled to the electronic medical record database 102 via the interface engine 120. In such an implementation, the patient data from the electronic medical record database 102 may be received through an EMR Application Programming Interface (EMR API). For example, the patient data from the electronic medical record database 102 may be received through the EMR API in a Fast Healthcare Interoperability Resources (FHIR) protocol. In such an implementation, the interface engine 120 may include a FHIR engine.

In operation, the interface engine 120 unwrap the patient data from the electronic medical record database 102 and send the patient data to the electronic medical record parser 122. The electronic medical record parser 122 may search the patient data for relevant data. For example, the electronic medical record parser 122 may perform such a search in response to a request from a main platform 130. For example, the main platform 130 may be centralized or may be distributed and may include some or all elements and components of one or more computers or computer systems. If relevant patient data is found, the electronic medical record parser 122 may send the relevant patient data to the main platform 130. If relevant patient data is found, the electronic medical record parser 122 may forward the request (e.g., after a period of time) from to the main platform 130 to the electronic medical record dispatch engine 124.

For example, the electronic medical record parser 122 may determine whether supplemental entry of current patient data is needed based on existing patient data in the electronic medical record database 102. In response to a determination that supplemental entry of current patient data is needed, the electronic medical record dispatch engine 124 may transfer a notification that the supplemental entry of the current patient data is needed to a user interface 126 associated with a care provider 128. For example, the electronic medical record dispatch engine 124 may send the notification to a nursing station or directly a caregiver's tablet, mobile organizer, the like, and/or combinations thereof. In some implementations, the user interface 126, may be implemented via a patient dashboard, a portable display (e.g., caregiver's tablet and/or mobile organizer), a workstation (e.g., at a nursing station), a medical imaging device display, a patient monitor display, a therapeutic device display, a medical management device display, the like, and/or combinations thereof.

As will be described in greater detail below, several different techniques of triggering notifications (e.g., an alert, a reminder, and/or a specific timeline) may be implemented in the electronic medical record dispatch engine 124. Additionally, or alternatively, two or more of such triggering techniques may be used concurrently by the electronic medical record dispatch engine 124. For example, the electronic medical record dispatch engine 124 may use different triggering techniques for different types of information. In one example, a frequency-based dispatching triggering technique for medication data while an algorithm-based dispatching triggering technique may be used for lab test results.

In some implementations, the EMR management system 100 may be utilized as a control point element of an Integrated Clinical Environment (ICE). As used herein, the “Integrated Clinical Environment (ICE)” refers to a platform to create a medical Internet of Things (IoT) associated with the care of a patient. In such an implementation, the EMR management system 100 may support many real-time clinical decision support algorithm and closed loop control algorithms of medical devices in the ICE.

In some implementations, the determination by the electronic medical record parser 122 of whether supplemental entry of the current patient data is needed may be based on one or more factors. For example, the determination of whether supplemental entry of the current patient data is needed may be based on a determination that at least a portion of the existing patient data is incorrect (e.g., a determination that the data has a high likely hood of being incorrect or the like). In another example, the determination of whether supplemental entry of the current patient data is needed may be based on a determination that at least a portion of the existing patient data is unavailable (e.g., missing, inaccessible, or the like). In still another example, the determination of whether supplemental entry of the current patient data is needed may be based on a determination that at least a portion of the existing patient data is required and unavailable. In a further example, the determination of whether supplemental entry of the current patient data is needed may be based on a determination that at least a portion of the existing patient data is expected and unavailable. In a still further example, the determination of whether supplemental entry of the current patient data is needed may be based on a determination that at least a portion of the existing patient data is stale based on a recurring fixed timer. Additionally, or alternatively, the determination of whether supplemental entry of the current patient data is needed may be based on a determination that at least a portion of the existing patient data is stale based on an error threshold.

Additionally, or alternatively, the electronic medical record parser 122 may receive a request from the clinical decision support applications 136 for supplemental entry of the current patient data in response to returned relevant patient data from the electronic medical record parser 122. Such a request from the clinical decision support applications 136 for supplemental entry of the current patient data may be based on the same or similar factors as described above with respect to determinations by the electronic medical record parser 122.

In the illustrated example, a first application 132 though an Nth application 134, which may include one or more clinical decision support applications 136, may be associated with the main platform 130. In such an example, the electronic medical record parser 122 may receive one or more patient data queries from one or more of the clinical decision support applications 136. The electronic medical record parser 122 may parse the electronic medical record database 102 for relevant patient data corresponding to the one or more patient data queries. The electronic medical record parser 122 may return the relevant patient data from the electronic medical record database 102 to the one or more clinical decision support applications 136.

In some implementations the returned relevant patient data may include estimated patient data acquired in response to a determination that the supplemental entry of current patient data is needed. Such estimated patient data may be supplied by a simulated database. Such a simulated database may utilize digital twin technology to perform the estimation, for example. In such an example, the estimated patient data may be marked to indicate its estimated nature (rather than measured patient data). Additionally, or alternatively, a weight factor may be applied to the estimated patient data so that the estimated patient data may have a lower weight than corresponding measured patient data.

In the illustrated example, the electronic medical record parser 122 may receive one or more settings from the one or more clinical decision support applications 136 for a recurring fixed timer as part of the one or more patient data queries. In such an example, the determination of whether supplemental entry of the current patient data is needed may include a determination that at least a portion of the existing patient data is stale based on the recurring fixed timer.

In some implementations, the electronic medical record parser 122 may receive one or more settings from the one or more clinical decision support applications 136 for an error threshold as part of the one or more patient data queries. In such an example, the determination of whether supplemental entry of the current patient data is needed may include a determination that at least a portion of the existing patient data is stale based on the error threshold.

Additionally, or alternatively, the electronic medical record parser 122 may receive a request from the clinical decision support applications 136 for supplemental entry of the current patient data in response to returned relevant patient data from the electronic medical record parser 122. Such a request from the clinical decision support applications 136 for supplemental entry of the current patient data may be based on the same or similar factors as described above with respect to determinations by the electronic medical record parser 122.

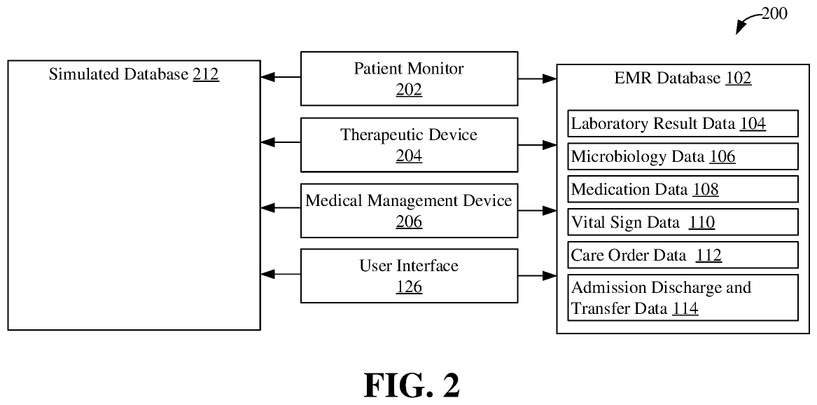

FIG. 2 is an illustration of a block diagram of a data entry system 200 according to an embodiment. In the illustrated example, the data entry system 200 may include a patient monitor 202, a therapeutic device 204, a medical management device 206, a simulated database 212, a user interface 126, the like (e.g., a medical imaging device), and or combinations thereof. For example, the patient monitor 202, the therapeutic device 204, the medical management device 206, and the user interface 126 may be in communication with the electronic medical record database 102 and/or with the simulated database 212 to deliver patient data.

In an example, the patient monitor 202 may be utilized to enter patient data to the electronic medical record database 102. For example, the patient monitor 202 may determine measured patient data. In such an example, the patient monitor 202 may be configured to monitor a patient for vital signs and the like, and the patient monitor 202 may communicate such measured patient data to the electronic medical record database 102.

In another example, the therapeutic device 204 may be utilized to enter patient data to the electronic medical record database 102. For example, the therapeutic device 204 may determine measured patient data. In such an example, the therapeutic device 204 may be configured to monitor the delivery of a particular therapy (e.g., a non-medication treatment) to a patient and may communicate such measured patient data to the electronic medical record database

In a further example, the medical management device 206 may be utilized to enter patient data to the electronic medical record database 102. For example, the medical management device 206 may determine measured patient data. In such an example, the medical management device 206 the medical management device 206 may be configured to monitor medication delivery to a patient and may communicate such measured patient data to the electronic medical record database 102.

Additionally, or alternatively, in a still further example, the user interface 126 may be utilized to enter patient data to the electronic medical record database 102. For example, a care provider may determine measured patient data through an analog device, a non-networked patient monitor, a non-networked therapeutic device, a non-networked medical management device, the like, and/or combinations thereof. In such an example, the care provider may enter such measured patient data to the electronic medical record database 102 via one or more user interfaces 126.

In some implementations, the simulated database 212 may generate estimated patient data. For example, the simulated database 212 utilize some measured patient data from the patient monitor 202, the therapeutic device 204, the medical management device 206, and/or the user interface 126 to generate some other estimated patient data. As described above, such a simulated database 212 may utilize digital twin technology to perform the estimation, for example. In such an example, such estimated patient data may be marked to indicate its estimated nature (rather than measured patient data). Additionally, or alternatively, a weight factor may be applied to the estimated patient data so that the estimated patient data may have a lower weight than corresponding measured patient data.

Some or all of the components of the data entry system 200 may be incorporated into the EMR management system 100 (FIG. 1), already discussed. Additionally, or alternatively, some or all of the components of the data entry system 200 may be in communication with the EMR management system 100 (FIG. 1).

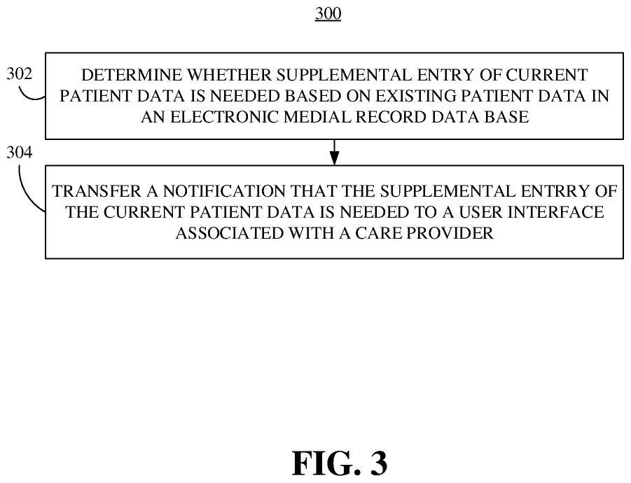

FIG. 3 shows an example method 300 for operating the EMR management system according to an embodiment. The method 300 may generally be implemented in an EMR management system, such as, for example, the EMR management system 100 (FIG. 1), already discussed.

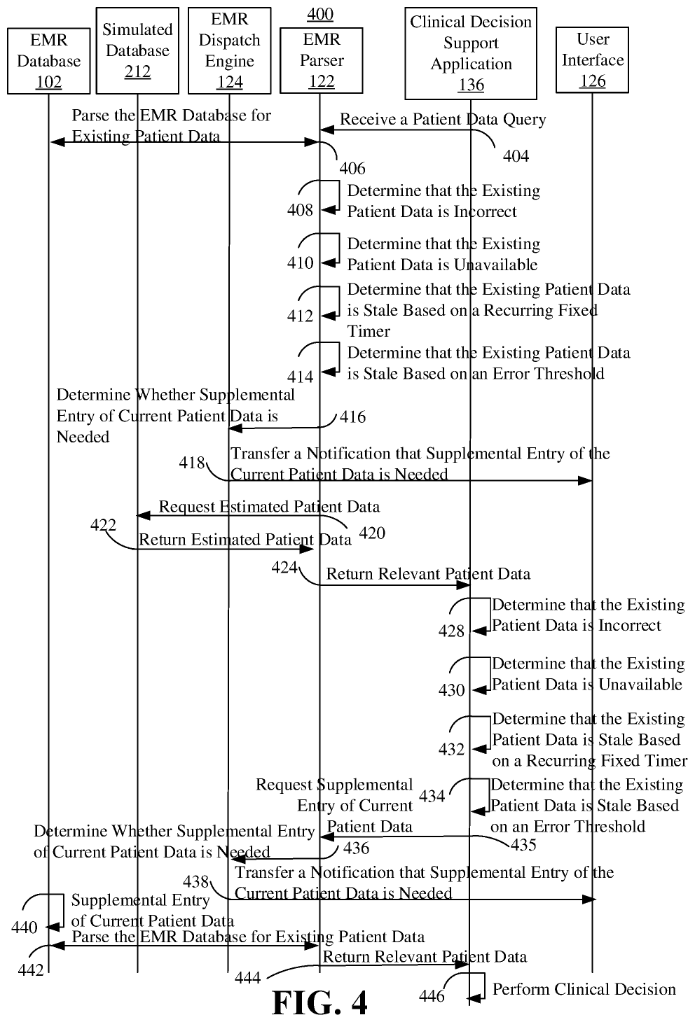

In an embodiment, the method 300 (as well as method 400 (FIG. 4) may be implemented in logic instructions (e.g., software), configurable logic, fixed-functionality hardware logic, etc., or any combination thereof. While certain portions of an EMR management system are illustrated in method 300, other portions of the EMR management system 100 (FIG. 1) have been intentionally left out to simplify the explanation of the method.

Illustrated processing block 302 provides for determining whether supplemental entry of current patient data is needed based on existing patient data. For example, an electronic medical record parser may determine whether supplemental entry of current patient data is needed based on existing patient data in an electronic medical record database.

Illustrated processing block 304 provides for transferring a notification that the supplemental entry of the current patient data is needed. For example, an electronic medical record dispatch engine may transfer the notification that the supplemental entry of the current patient data is needed to a user interface associated with a care provider.

In operation, the method 300 may be used to notify responsible operators and/or clinicians about patient information that is missing and/or overdue in the EMR system. Such a notification may be generated based on one or more of several different criteria.

Additionally, or alternatively, in some implementations, the determination by the electronic medical record parser of whether supplemental entry of the current patient data is needed may be based on one or more techniques.

In one example, the determination by the electronic medical record parser of whether supplemental entry of the current patient data is needed may utilize a frequency-based technique. In such an example a notification (e.g., an alert and/or a specific timeline) may be sent when certain data is missing after certain period of time.

For example, a frequency-based notification may be used to generate a reminder if a certain numeric is not provided in the EMR database after a fixed period of time. In such an implementation, the patient might need to be evaluated for the pain score every twelve hours. If this score is not provided after six hours, the electronic medical record dispatch engine may send a notification of a request for this data to a nurse station. Additionally, this information may also be used as protocol compliance information.

In another example, the determination by the electronic medical record parser of whether supplemental entry of the current patient data is needed may utilize an active application-based technique. For example, the notification may be sent when certain data is needed by at least one active application. In such an example, a frequency of supplying refreshing the data may also be set by the individual active applications.

For example, an active application-based notification may be used to generate a reminder. In such an implementation, the generation of a reminder related to the requested data may be done by a running active application. For example, even a frequency of patient data collection may be set by the active application. The care facility may then follow the request of data by the active application depending on a determined importance of the requested patient data to the active application. For example, an active application may need non-invasive blood pressure every six hours to calculate an optimum rate of a particular vasodilator drug. In such an example, if a clinician trusts the result of the active application, the clinician may ask the nurses to frequently make such measurements and enter the updated patient data the system; and, while the active application is running, if the updated patient data is not provided within the prescribed six hours, the system may dispatch a notification to a nursing station, a patient monitor, the like, and/or combinations thereof.

Additionally, or alternatively, in a further example, the determination by the electronic medical record parser of whether supplemental entry of the current patient data is needed may utilize an algorithm-based technique. For example, the notification may be sent based on a forecasted need for new patient data and/or refreshed patient data. In such an example, the forecasted need for new patient data and/or refreshed patient may be based on one or more algorithms. These algorithms may determine that a particular CDS algorithm currently needs such patient data and/or forecast that the particular CDS algorithm will need such patient data in the future. For example, a blood gas estimation-type algorithm may determine an estimation error progress overtime. When such an estimation error is beyond some value (e.g., a threshold value), a request for new patient data and/or refreshed patient data is generated.

For example, an algorithm-based notification may be used to generate a reminder. In such an implementation, the generation of a reminder related to the requested data may be done when the algorithm decides new patient data and/or refreshed patient data is needed to ensure the reliability of the output results. For example, a time between refreshed measurements may change in response to changes in the analysed data from the patient (e.g., and as long as the active application does not require an expected value). In one example, consider an active application that might be estimating a blood gas and an acidity of a patient to suggest a particular ventilator and oxygen therapy setting. In such an example, an upper bound for the estimation error may also be calculated. Depending on the condition of the patient, such an upper bound may change. In such a situation, when the change in the upper bound reaches a threshold, the algorithm may suggest a new patient data gathering event (e.g., a lab test to get a ground truth for the blood gas). The system may then generate a notification that a blood gas test is needed.

Additional and/or alternative operations for method 300 are described in greater detail below in the description of FIG. 4.

FIG. 4 is a flowchart of an example of another method 400 for operating the EMR management system according to an embodiment. The method 400 may generally be implemented in an EMR management system, such as, for example, the EMR management system 100 (FIG. 1), already discussed.

Illustrated processing block 404 provides for receiving a patient data query. For example, the electronic medical record parser 122 may receive one or more patient data queries from one or more of the clinical decision support applications 136.

Illustrated processing block 406 provides for parsing the electronic medical record database 102 for existing patient data. For example, the electronic medical record parser 122 may parse the electronic medical record database 102 for relevant patient data corresponding to the one or more patient data queries.

Referring to processing blocks 408-414, in some implementations, the reception, from the one or more clinical decision support applications 136, of the request for supplemental entry of the current patient data may be based on one or more factors.

Illustrated processing block 408 provides for determining that the existing patient data is incorrect. For example, the determination of whether supplemental entry of the current patient data is needed may be based on a determination by the electronic medical record parser 122 that at least a portion of the existing patient data is incorrect (e.g., a determination that the data has a high likely hood of being incorrect or the like).

Illustrated processing block 410 provides for determining that the existing patient data is unavailable. In some implementations, processing block 430 may also provide for a determination that at least a portion of the existing patient data is required and unavailable and/or a determination that at least a portion of the existing patient data is expected and unavailable. For example, the determination of whether supplemental entry of the current patient data is needed may be based on a determination by the electronic medical record parser 122 that at least a portion of the existing patient data is unavailable (e.g., missing, inaccessible, or the like). Additionally, or alternatively, the determination of whether supplemental entry of the current patient data is needed may be based on a determination by the electronic medical record parser 122 that at least a portion of the existing patient data is required and unavailable and/or expected and unavailable.

Illustrated processing block 412 provides for determining that the existing patient data is stale based on a recurring fixed timer. For example, the determination of whether supplemental entry of the current patient data is needed may be based on a determination by the electronic medical record parser 122 that at least a portion of the existing patient data is stale based on a recurring fixed timer. In some implementations, the electronic medical record parser 122 may receive one or more settings from the one or more clinical decision support applications 136 for a recurring fixed timer as part of the one or more patient data queries. In such an example, the determination of whether supplemental entry of the current patient data is needed may include a determination that at least a portion of the existing patient data is stale based on the recurring fixed timer.

Illustrated processing block 414 provides for determining that the existing patient data is stale based on an error threshold. For example, the determination of whether supplemental entry of the current patient data is needed may be based on a determination by the electronic medical record parser 122 that at least a portion of the existing patient data is stale based on an error threshold. In some implementations, the electronic medical record parser 122 may receive one or more settings from the one or more clinical decision support applications 136 for an error threshold as part of the one or more patient data queries. In such an example, the determination of whether supplemental entry of the current patient data is needed may include a determination that at least a portion of the existing patient data is stale based on the error threshold.

Illustrated processing block 416 provides for determining whether supplemental entry of current patient data is needed. For example, the electronic medical record parser 122 may determine whether supplemental entry of current patient data is needed based at least in part on the operations of blocks 408-414.

Illustrated processing block 418 provides for transferring a notification that supplemental entry of the current patient data is needed. For example, the electronic medical record parser 122 may transfer a notification that supplemental entry of the current patient data is needed to one or more user interfaces 126.

Illustrated processing block 420 provides for requesting estimated patient data. For example, the electronic medical record parser 122 may request estimated patient data from the simulated database 212.

Illustrated processing block 422 provides for returning estimated patient data. For example, the simulated database 212 may return the estimated patient data to the electronic medical record parser 122.

Illustrated processing block 424 provides for returning relevant patient data. For example, the electronic medical record parser 122 may return the returned relevant patient data from the electronic medical record database 102 to the one or more clinical decision support application 136. In some implementations the returned relevant patient data may include estimated patient data acquired in response to a determination that the supplemental entry of current patient data is needed. Such estimated patient data may be supplied by the simulated database 212. The simulated database 212 may utilize digital twin technology to perform the estimation, for example. In such an example, the estimated patient data may be marked to indicate its estimated nature (rather than measured patient data). Additionally, or alternatively, a weight factor may be applied to the estimated patient data so that the estimated patient data may have a lower weight than corresponding measured patient data.

Referring to processing blocks 428-434, in some implementations, the determination by the electronic medical record parser 122 of whether supplemental entry of the current patient data is needed may be based on one or more factors.

Illustrated processing block 428 provides for determining that the existing patient data is incorrect. For example, the determination of whether supplemental entry of the current patient data is needed may be based on a determination by the clinical decision support application 136 that at least a portion of the existing patient data is incorrect (e.g., a determination that the data has a high likely hood of being incorrect or the like).

Illustrated processing block 430 provides for determining that the existing patient data is unavailable. In some implementations, processing block 430 may also provide for a determination that at least a portion of the existing patient data is required and unavailable and/or a determination that at least a portion of the existing patient data is expected and unavailable. For example, the determination of whether supplemental entry of the current patient data is needed may be based on a determination by the clinical decision support application 136 that at least a portion of the existing patient data is unavailable (e.g., missing, inaccessible, or the like). Additionally, or alternatively, the determination of whether supplemental entry of the current patient data is needed may be based on a determination by the clinical decision support application 136 that at least a portion of the existing patient data is required and unavailable and/or expected and unavailable.

Illustrated processing block 432 provides for determining that the existing patient data is stale based on a recurring fixed timer. For example, the determination of whether supplemental entry of the current patient data is needed may be based on a determination by the clinical decision support application 136 that at least a portion of the existing patient data is stale based on a recurring fixed timer.

Illustrated processing block 434 provides for determining that the existing patient data is stale based on an error threshold. For example, the determination of whether supplemental entry of the current patient data is needed may be based on a determination by the clinical decision support application 136 that at least a portion of the existing patient data is stale based on an error threshold.

Illustrated processing block 435 provides for requesting supplemental entry of current patient data. For example, the electronic medical record parser 122 may receive a request from the clinical decision support applications 136 for supplemental entry of the current patient data in response to returned relevant patient data from the electronic medical record parser 122. Such a request from the clinical decision support applications 136 for supplemental entry of the current patient data may be based on the same or similar factors as described above with respect to determinations by the electronic medical record parser 122.

Illustrated processing block 436 provides for determining whether supplemental entry of current patient data is needed. For example, the electronic medical record parser 122 may determine whether supplemental entry of current patient data is needed based at least in part on the operations of blocks 428-434.

Illustrated processing block 438 provides for transferring a notification that supplemental entry of the current patient data is needed. For example, the electronic medical record dispatch engine 124 may transfer a notification that supplemental entry of the current patient data is needed to one or more of the user interfaces 126.

Illustrated processing block 440 provides for supplemental entry of current patient data. For example, supplemental entry of current patient data may be made to data stored in the electronic medical record database 102.

Illustrated processing block 442 provides for parsing the electronic medical record database 102 for existing patient data. For example, the electronic medical record parser 122 may parse the electronic medical record database 102 for existing patient data after the supplemental entry of current patient data.

Illustrated processing block 444 provides for returning relevant patient data. For example, the electronic medical record parser 122 may return relevant patient data from the electronic medical record database 102 to the one or more clinical decision support application 136 in response to the supplemental entry of current patient data.

Illustrated processing block 446 provides for performing a clinical decision. For example, the clinical decision support application 136 may perform a clinical decision based at least in part on the current patient data.

It will be appreciated that some or all of the operations in method 400 above that have been described using a “pull” architecture (e.g., polling for new information followed by a corresponding response) may instead be implemented using a “push” architecture (e.g., sending such information when there is new information to report), and vice versa.

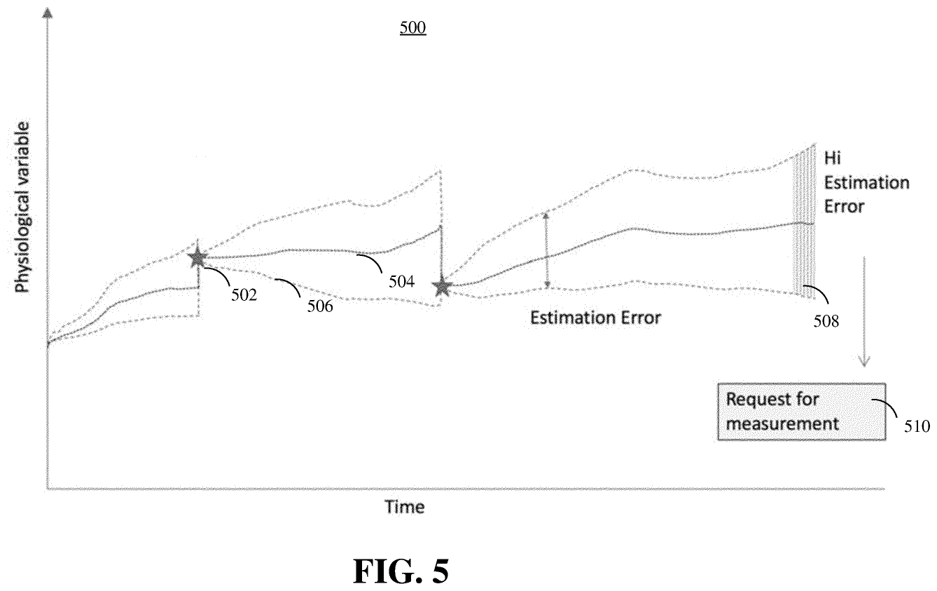

FIG. 5 shows an illustrative screen shot 500 of the EMR management system 100 (FIG. 1). In the illustrated example, the screen shot 500 shows a notification for display on a user interface that has been sent in response to an algorithm based request for new or refreshed patient data.

In some implementations, the screen shot 500 may include a graphical indication and/or a text indication of measured patient data 502, estimated patient data 504, upper and lower estimation bounds 506, an alert of there being a high estimation error 508, a request for a measurement 510, the like, and/or combinations thereof. In the illustrated example, the screen shot 500 shows the measured patient data 502 illustrated with a star symbol. The estimated patient data 504 is illustrated with a solid line located between adjacent star symbols. The upper and lower estimation bounds 506 is illustrated with a dashed pair of lines on either side of the estimated patient data 504. The alert of there being a high estimation error 508 is illustrated with a graphical indication (e.g., a shaded area between upper and lower estimation bounds 506) and/or a text indication of the alert. The request for a measurement 510 is illustrated with a text indication of the request.

In operation, such a notification may provide a visual context to the clinician to understand the importance of gathering and maintaining timely patient data. In the illustrated example, the screen shot 500 may provide visual feedback for the clinicians. The solid line of the estimated patient data 504 shows the estimate value of a physiological variable. Since this is an estimation, there may also an error associated to this variable. The dashed lines of the upper and lower estimation bounds 506 may show an error range of such an estimation. As illustrated, the estimation error may increase over time until a new measured patient data 502 reference value is provided. The measured patient data 502 star points may illustrate times when a new measured value is provided to the algorithm. In operation, such a new measured patient data 502 value may be provided from an EMR database to an analytics platform of an electronic medical record parser. When such a new measured patient data 502 value is received by the electronic medical record parser, the estimation error range of the upper and lower estimation bounds 506 may reduce to zero. The visualization of the screen shot 500 may be used for the clinicians to decide when it is time to take a new test. Additionally, or alternatively, an algorithm and/or active application may automatically generate a notification (e.g., a request for such a new test) when the estimation error range of the upper and lower estimation bounds 506 increases beyond a threshold.

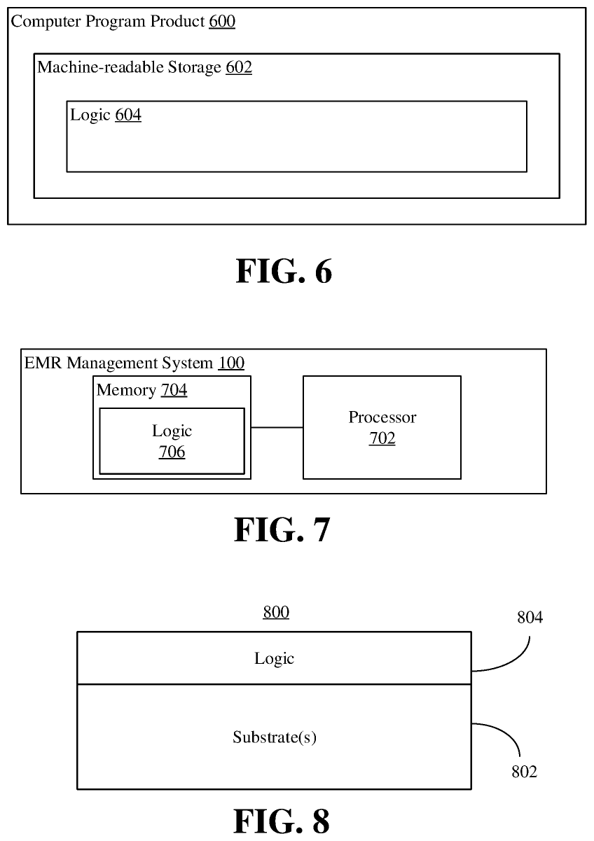

FIG. 6 illustrates a block diagram of an example computer program product 600. In some examples, as shown in FIG. 6, computer program product 600 includes a machine-readable storage 602 that may also include logic 604. In some implementations, the machine-readable storage 602 may be implemented as a non-transitory machine-readable storage. In some implementations the logic 604 may be implemented as machine-readable instructions, such as software, for example. In an embodiment, the logic 604, when executed, implements one or more aspects of the method 300 (FIG. 3), the method 400 (FIG. 4), and/or realize the EMR management system 100 (FIG. 1), already discussed.

FIG. 7 shows an illustrative example of the EMR management system 100. In the illustrated example, the EMR management system 100 may include a processor 702 and a memory 704 communicatively coupled to the processor 702. The memory 704 may include logic 706 as a set of instructions. In some implementations the logic 706 may be implemented as software. In an embodiment, the logic 706, when executed by the processor 702, implements one or more aspects of the method 300 (FIG. 3), the method 400 (FIG. 4), and/or realize the EMR management system 100 (FIG. 1), already discussed.

In some implementations, the processor 702 may include a general purpose controller, a special purpose controller, a storage controller, a storage manager, a memory controller, a micro-controller, a general purpose processor, a special purpose processor, a central processor unit (CPU), the like, and/or combinations thereof.

Further, implementations may include distributed processing, component/object distributed processing, parallel processing, the like, and/or combinations thereof. For example, virtual computer system processing may implement one or more of the methods or functionalities as described herein, and the processor 702 described herein may be used to support such virtual processing.

In some examples, the memory 704 is an example of a computer-readable storage medium. For example, memory 704 may be any memory which is accessible to the processor 702, including, but not limited to RAM memory, registers, and register files, the like, and/or combinations thereof. References to “computer memory” or “memory” should be interpreted as possibly being multiple memories. The memory may for instance be multiple memories within the same computer system. The memory may also be multiple memories distributed amongst multiple computer systems or computing devices.

FIG. 8 shows an illustrative semiconductor apparatus 800 (e.g., chip and/or package). The illustrated apparatus 800 includes one or more substrates 802 (e.g., silicon, sapphire, or gallium arsenide) and logic 804 (e.g., configurable logic and/or fixed-functionality hardware logic) coupled to the substrate(s) 802. In an embodiment, the logic 804 implements one or more aspects of the method 300 (FIG. 3), the method 400 (FIG. 4), and/or realize the EMR management system 100 (FIG. 1), already discussed.

In some implementations, logic 804 may include transistor array and/or other integrated circuit/IC components. For example, configurable logic and/or fixed-functionality hardware logic implementations of the logic 804 may include configurable logic such as, for example, programmable logic arrays (PLAs), field programmable gate arrays (FPGAs), complex programmable logic devices (CPLDs), or fixed-functionality logic hardware using circuit technology such as, for example, application specific integrated circuit (ASIC), complementary metal oxide semiconductor (CMOS) or transistor-transistor logic (TTL) technology, the like, and/or combinations thereof.

All definitions, as defined and used herein, should be understood to control over dictionary definitions, definitions in documents incorporated by reference, and/or ordinary meanings of the defined terms.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. The term “coupled” may be used herein to refer to any type of relationship, direct or indirect, between the components in question, and may apply to electrical, mechanical, fluid, optical, electromagnetic, electromechanical, or other connections. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components.

In the claims, as well as in the specification above, the terms “first”, “second”, etc. may be used herein only to facilitate discussion, and carry no particular temporal or chronological significance unless otherwise indicated.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The indefinite articles “a” and “an,” as used herein in the specification and in the claims, unless clearly indicated to the contrary, should be understood to mean “at least one.”

As used herein, the term “or” or “and/or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used in this application and in the claims, a list of items joined by the term “one or more of” may mean any combination of the listed terms. For example, the phrases “one or more of A, B or C” may mean A; B; C; A and B; A and C; B and C; or A, B and C.

As is described above in greater detail, one or more processor, other unit, the like, and/or combinations thereof may fulfill the functions of several items recited in the claims.

As is described above in greater detail, a computer program may be stored/distributed on a suitable medium, such as an optical storage medium or a solid-state medium supplied together with or as part of other hardware, but may also be distributed in other forms, such as via the Internet or other wired or wireless telecommunication systems.

It should also be understood that, unless clearly indicated to the contrary, in any methods discussed herein that include more than one step or act, the order of the steps or acts of the method is not necessarily limited to the order in which the steps or acts of the method are recited. Further, such methods may include additional or alternative steps or acts.

As used in the claims, the mere fact that certain measures are recited in mutually different dependent claims does not indicate that a combination of these measured cannot be used to advantage.

It is also noted that the claims may include reference signs/numerals in accordance with PCT Rule 6.2 (b). However, the present claims should not be considered to be limited to the exemplary embodiments corresponding to the reference signs/numerals.

Those skilled in the art will appreciate from the foregoing description that the broad techniques of the embodiments of the present invention can be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

1. An apparatus, comprising:
an electronic medical record parser communicatively coupled to an electronic medical record database, the electronic medical record parser to determine whether supplemental entry of current patient data is needed based on existing patient data in the electronic medical record database; and
an electronic medical record dispatch engine communicatively coupled to the electronic medical record parser, the electronic medical record dispatch engine to transfer a notification that the supplemental entry of the current patient data is needed to a user interface associated with a care provider.

2. The apparatus of claim 1, wherein determination of whether supplemental entry of the current patient data is needed is based on one or more of:
a determination that at least a portion of the existing patient data is incorrect,
a determination that at least a portion of the existing patient data is unavailable,
a determination that at least a portion of the existing patient data is required and unavailable,
a determination that at least a portion of the existing patient data is expected and unavailable,
a determination that at least a portion of the existing patient data is stale based on a recurring fixed timer, or
a determination that at least a portion of the existing patient data is stale based on an error threshold.

3. The apparatus of claim 1, wherein the electronic medical record parser is further to:
receive one or more patient data queries from one or more clinical decision support applications,
parse the electronic medical record database for relevant patient data corresponding to the one or more patient data queries, and
return the relevant patient data from the electronic medical record database to the one or more clinical decision support applications,
wherein returned relevant patient data includes estimated patient data acquired in response to a determination that the supplemental entry of current patient data is needed.

4. The apparatus of claim 3, wherein the electronic medical record parser is further to:
receive, from the one or more clinical decision support applications, one or more settings for a recurring fixed timer as part of the one or more patient data queries, and
wherein the determination of whether supplemental entry of the current patient data is needed comprises a determination that at least a portion of the existing patient data is stale based on the recurring fixed timer.

5. The apparatus of claim 3, wherein the electronic medical record parser is further to:
receive, from the one or more clinical decision support applications, one or more settings for an error threshold as part of the one or more patient data queries, and
wherein the determination of whether supplemental entry of the current patient data is needed comprises a determination that at least a portion of the existing patient data is stale based on the error threshold.

6. The apparatus of claim 1, wherein the electronic medical record parser is further to:
receive one or more patient data queries from one or more clinical decision support applications,
parse the electronic medical record database for relevant patient data corresponding to the one or more patient data queries,
return the relevant patient data from the electronic medical record database to the one or more clinical decision support applications, and
receive, from the one or more clinical decision support applications, a request for supplemental entry of the current patient data.

7. The apparatus of claim 6, wherein reception, from the one or more clinical decision support applications-436), of the request for supplemental entry of the current patient data is based on one or more of:
a determination that at least a portion of the existing patient data is incorrect,
a determination that at least a portion of the existing patient data is unavailable,
a determination that at least a portion of the existing patient data is required and unavailable,
a determination that at least a portion of the existing patient data is expected and unavailable,
a determination that at least a portion of the existing patient data is stale based on a recurring fixed timer, or
a determination that at least a portion of the existing patient data is stale based on an error threshold.

8. A method, comprising:
determining, via an electronic medical record parser, whether supplemental entry of current patient data is needed based on existing patient data in an electronic medical record database; and
transferring, via an electronic medical record dispatch engine, a notification that the supplemental entry of the current patient data is needed to a user interface associated with a care provider.

9. The method of claim 8, wherein determination of whether supplemental entry of the current patient data is needed is based on one or more of:
determining that at least a portion of the existing patient data is incorrect,
determining that at least a portion of the existing patient data is unavailable,
a determination that at least a portion of the existing patient data is required and unavailable,
a determination that at least a portion of the existing patient data is expected and unavailable,
determining that at least a portion of the existing patient data is stale based on a recurring fixed timer, or
determining that at least a portion of the existing patient data is stale based on an error threshold.

10. The method of claim 8, further comprising:
receiving, via the electronic medical record parser, one or more patient data queries from one or more clinical decision support applications,
parsing, via the electronic medical record parser, the electronic medical record database for relevant patient data corresponding to the one or more patient data queries, and
returning, via the electronic medical record parser, the relevant patient data from the electronic medical record database to the one or more clinical decision support applications,
wherein returned relevant patient data includes estimated patient data acquired in response to a determination that the supplemental entry of current patient data is needed.

11. The method of claim 10, further comprising:
receiving, from the one or more clinical decision support applications, one or more settings for a recurring fixed timer as part of the one or more patient data queries, and
wherein the determination of whether supplemental entry of the current patient data is needed comprises a determination that at least a portion of the existing patient data is stale based on the recurring fixed timer.

12. The method (300) of claim 10, further comprising:
receiving, from the one or more clinical decision support applications, one or more settings for an error threshold as part of the one or more patient data queries, and
wherein the determination of whether supplemental entry of the current patient data is needed comprises a determination that at least a portion of the existing patient data is stale based on the error threshold.

13. The method of claim 8, further comprising:
receiving, via the electronic medical record parser, one or more patient data queries from the one or more clinical decision support applications,
parsing, via the electronic medical record parser, the electronic medical record database for relevant patient data corresponding to the one or more patient data queries,
returning, via the electronic medical record parser, the relevant patient data from the electronic medical record database to the one or more clinical decision support applications, and
receiving, from the one or more clinical decision support applications, a request for supplemental entry of the current patient data.

14. The method of claim 13, wherein reception, from the one or more clinical decision support applications, of the request for supplemental entry of the current patient data is based on one or more of:
determining that at least a portion of the existing patient data is incorrect,
determining that at least a portion of the existing patient data is unavailable,
a determination that at least a portion of the existing patient data is required and unavailable,
a determination that at least a portion of the existing patient data is expected and unavailable,
determining that at least a portion of the existing patient data is stale based on a recurring fixed timer, or
determining that at least a portion of the existing patient data is stale based on an error threshold.

15. A machine-readable storage including machine-readable instructions, which when executed, implement the method or realize the apparatus according to claim 1.

16. An apparatus, comprising:
means for performing the method according to claim 8.
